(19) United States
(12) Patent Application Publication
(10) Pub. No.: US 2026/0232583 A1
(43) Pub. Date: Aug. 13, 2026

(54) FLUID COMPOSITIONS FOR FLUID BALANCE

(71) Applicant: Stokely-Van Camp, Inc., Chicago, IL (US)

(72) Inventors: Lindsay Baker, Valhalla, NY (US); Brian Song, Valhalla, NY (US); Krishnan Subramanian, Mahwah, NJ (US); Kiran Annapragada, Chicago, IL (US); Yuan Fang, Valhalla, NY (US); Rohil Bhatnagar, Valhalla, NY (US); Aidee Penaloza, Valhalla, NY (US); Kathlene A McManus, Valhalla, NY (US); Mara Calderon Guerra, White Plains, NY (US); Grace Feng, Valhalla, NY (US)

(21) Appl. No.: 19/530,024

(22) Filed: Feb. 4, 2026

Related U.S. Application Data
(60) Provisional application No. 63/755,639, filed on Feb. 7, 2025.

(51) Int. Cl. A61K 9/00; A23L 2/38; A23L 2/60; A23L 2/62; A23L 33/16; A61K 33/00; A61K 47/10; A61K 47/26

(52) U.S. Cl. CPC A61K 9/0095; A23L 2/38; A23L 2/60; A23L 2/62; A23L 33/16; A61K 33/00; A61K 47/10; A61K 47/26

(57) ABSTRACT

Fluid balance concentrates that include glycerol, a clouding agent, and water are described together with ready-to-drink fluid compositions that include the fluid balance concentrate. Other ready-to-drink fluid compositions that do not include the fluid balance concentrate and/or clouding agent are also described.

This application claims priority to U.S. Application No. 63/755,639 filed Feb. 7, 2025, the entire contents of which are incorporated herein by reference.

The following disclosure describes improved fluid balance concentrates (also may be referred to as euhydration concentrates) and ready-to-drink fluid compositions that include the improved fluid balance concentrates and their use to provide rapid hydration and/or to maintain and/or to improve bodily fluid balance, i.e., euhydration, particularly as compared to water alone (i.e., pure water). The following disclosure also describes other aspects of ready-to-drink fluid compositions that may not include the fluid balance concentrate.

### BACKGROUND

Water is a large constituent of the human body and is important for optimal physiological function and health. Drinking appropriate volumes of fluid throughout the day is important to maintain adequate hydration levels. However, another important factor in hydration is the composition of fluid consumed. To maintain overall whole-body fluid balance, it is important that ingested fluid is retained in the body instead of losing it through urination.

While there has been research into the use of electrolytic beverage compositions, including the use of such beverage compositions to achieve hyperhydration prior to exercise, there is a continuing need for beverages to aid in promoting fluid balance, i.e., euhydration for the general active population in situations other than prior to, during, or after exercising, i.e., at rest. Euhydration refers to a state of normal, optimal body water balance, where fluid levels are sufficient to support normal physiological functions, preventing both dehydration (hypohydration) and fluid overload (hyperhydration).

To achieve a state of euhydration, it would be desirable to provide a fluid composition that contains ingredients that may improve fluid balance (promote fluid retention) and simultaneously or separately provide rapid hydration.

### SUMMARY

To that end, the present inventors after thoroughly investigating discovered a fluid balance concentrate that comprises, consists essentially of, or consists of glycerol, a clouding agent, and water. In some embodiments, the fluid balance concentrate has a pH in a range of about 4.0 to about 4.5 and/or a water activity level less than 0.50 or from about 0.15 to about 0.49. Additionally, the fluid balance concentrate may, in some instances, exhibit a viscosity between about 30 cP to about 300 cP. In some embodiments, a 1% solution of the fluid balance concentrate may exhibit a turbidity in a range from about 100 Nephelometric turbidity units (NTU) to about 300 NTU.

It has been found that the above fluid balance concentrate may be useful in preparing fluid balance ready-to-drink fluid compositions (may also be referred to simply as ready-to-drink fluid compositions). Ready-to-drink refers to and means a fluid composition that can be consumed directly without any further preparation needed, like opening a can or bottle, essentially, it's ready to drink right out of the container. Such ready-to-drink fluid compositions may be useful for maintaining and/or improving bodily fluid balance, i.e., euhydration. To this end, without being bound by the following hypothesis, it is believed that the use of the described fluid balance concentrate may, when combined with electrolytes, (a) provide an improved mouthfeel, particularly when compared to a ready-to-drink electrolyte-containing fluid composition with no fluid balance concentrate, (b) provide an organoleptic perception of mouth coating, (c) provide a perception of decreased bitterness and sweetness that may linger from the presence of non-nutritive sweeteners, when present, and/or (d) provide a perception of decreased saltiness and/or decreased perception of minerals.

An exemplary ready-to-drink fluid composition may comprise, consist essentially of, or consist of water, an amount of the fluid balance concentrate to provide from about 0.1 wt. % to about 5.0 wt. % in the ready-to-drink composition, sodium, electrolytes other than sodium, and a sweetener. In some embodiments, the sweetener is a non-nutritive sweetener. In some instances, the fluid balance ready-to-drink fluid composition may be free of carbohydrates.

In other aspects, a ready-to-drink fluid composition may comprise, consist essentially of, or consist of water, glycerol, sodium, electrolytes other than sodium, and a sweetener. In some embodiments, the sweetener is a non-nutritive sweetener. In some instances, the ready-to-drink fluid composition may be free of carbohydrates. In accordance with this aspect, it has been found that, while the presence of sodium in a ready-to-drink fluid composition may enhance the palatability and may stimulate the physiological drive to drink, high sodium concentrations (i.e., sodium concentrations ≥50 mmol/L) may decrease drink palatability in the absence of glycerol, which could hinder ad libitum fluid intake.

Surprisingly, it has been found that a ready-to-drink fluid composition that contains both sodium and glycerol may require less sodium required to provide the same level of fluid balance as a ready-to-drink fluid composition that contains a greater amount of sodium but does not contain glycerol. For example, a ready-to-drink fluid composition that contains both an amount of glycerol and an amount of sodium (denoted as ready-to-drink fluid composition (a)) may be compared to an identical ready-to-drink fluid composition that contains a greater amount of sodium and does not contain glycerol, i.e., the glycerol has been replaced with additional sodium and water (denoted as fluid composition (b)). When the fluid balance of a human who has consumed ready-to-drink fluid composition (a) is compared to the fluid balance of the same human who has consumed ready-to-drink fluid composition (b), it has been found that to achieve the same fluid balance, the amount of sodium present in ready-to-drink fluid composition (a) may be less than about 2 mmol/L to about 10 mmol/L than the amount of sodium present in fluid composition (b).

Typically, the above ready-to-drink fluid compositions may be provided in a consumer-friendly size bottle. Such a bottle may contain from about 100 ml to about 3.8 liters, or about 100, 200, 250, 300, 400, 500, 600, 700, 750, 800, 900, 1,000, 1,500, 2000, 2,500, 3,000 or about 3,800 ml of the ready-to-drink fluid compositions. In some instances the bottle may contain about 828 ml (28 oz) of the ready-to-drink fluid compositions. The described ready-to-drink fluid compositions may provide superior rehydration, improved fluid retention, and reduced urine fluid loss. The described ready-to-drink fluid compositions may enhance rehydration and/or euhydration, for example, by improving voluntary fluid intake.

In accordance with the above, it is contemplated that the described ready-to-drink fluid compositions may be used in a method to provide rapid hydration and/or to maintain and/or to improve fluid balance and/or fluid retention in healthy, euhydrated humans. In some instances, the described methods provide a desired fluid balance and fluid retention for a period of time after ingestion of the described fluid compositions. In some embodiments, the period of time is greater than about 30 minutes and may be up to about 240 minutes. The method may include providing an amount of the ready-to-drink compositions described above and in the following to a healthy, euhydrated human.

All percentages used or recited in this description refer to a percentage by weight, unless specifically stated otherwise. Other aspects and advantages of this invention will be appreciated from the following detailed description.

The described compositions and methods extend to any novel aspects or described features. Furthermore, any, some and/or all features in one aspect can be applied to any, some and/or all features in any other aspect, in any appropriate combination. It should also be appreciated that particular combinations of the various features described and defined in any aspects of the invention can be implemented and/or supplied and/or used independently.

### DESCRIPTION OF THE DRAWINGS

The foregoing aspects and many of the attendant advantages of the present technology will become more readily appreciated by reference to the following Description, when taken in conjunction with the accompanying drawings.

FIG. 1 shows the Fluid Balance in subjects over time after ingestion of exemplary beverages by the subjects, as measured by the percentage change in body mass of the subject and as compared to a placebo.

FIG. 2 shows the Fluid Balance in subjects over time after ingestion of an exemplary beverage by the subjects, as measured by the percentage change in body mass of the subject and as compared to a placebo.

### DETAILED DESCRIPTION

### Concentrate

As noted above, a fluid balance concentrate comprises, consists essentially of, or consists of glycerol, a clouding agent, and water. In some embodiments, the fluid balance concentrate has a pH in a range of about 4.0 to about 4.5 and/or a water activity level less than 0.50 or from about 0.15 to about 0.49. Additionally, the fluid balance concentrate may, in some instances, exhibit a viscosity between about 30 cP to about 300 cP.

Glycerol, also known as 1,2,3-Propanetriol, is a colorless, odorless, viscous liquid that is sweet-tasting. Glycerol may be present in the fluid balance concentrate in an amount from about 82% to about 92% or about 82%, 83%, 84%, 85%, 86%, 87%, 88%, 89%, 90%, 91%, or 92%. To this end, the glycerol may be present in the fluid balance concentrate at a lower limit of about 82%, 83%, 84%, 85%, 86%, 87%, 88%, 89%, 90%, or about 91% and at a higher limit or about 83%, 84%, 85%, 86%, 87%, 88%, 89%, 90%, 91%, or about 92%, or any range that could be created from these ranges.

While glycerol is categorized by the U.S. Academy of Nutrition and Dietetics as a carbohydrate and the U.S. Food and Drug Administration (FDA) carbohydrate designation includes all caloric macronutrients excluding protein and fat, for purposes of this description and claims, glycerol is not considered to be a carbohydrate. Rather, it is a polyol, i.e., it has multiple hydroxyl groups. In some embodiments, the described fluid balance concentrate and fluid compositions made from the described fluid balance concentrate are free of carbohydrates.

The clouding agent may be any suitable component that will provide a cloudy appearance to the ready-to-drink fluid compositions. In addition, it appears that the presence of a clouding agent in combination with the glycerol and water positively modulates the desired viscosity of the fluid balance concentrate. Non-limiting examples of clouding agents include, but are not limited to, ester gum, gum Arabic, sucrose acetate isobutyrate (SAIB), starch components, and mixtures thereof. In some embodiments, the clouding agent is a starch component. In some embodiments the starch component may include retrograded amylose, retrograded maltodextrin, retrograded maltodextrin free of titanium dioxide, waxy maize starch, waxy maize-based emulsifiers, modified food starch.

The clouding agent may be included in the fluid balance concentrate in an amount from about 3.0% to about 8.0% or about 4.0%, 5.0%, 6.0%, 7.0%, or about 8.0%. To this end, the clouding agent may be present in the fluid balance concentrate at a lower limit of about 3.0%, 4.0%, 5.0%, 6.0%, or about 7.0%, and at a higher limit of about 4.0%, 5.0%, 6.0%, 7.0%, or about 8.0%, or any range that could be created from these ranges.

Water is included in the fluid balance concentrate in an amount from about 5.0% to about 20.0% or from about 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, or about 20.0%. In this regard, the water may be present at a lower limit of about 5.0%, 6.0%, 7.0%, 8.0%, 9.0%, 10.0%, 11.0%, 12.0%, 13.0%, 14.0%, 15.0%, 16.0%, 17.0%, 18.0%, or about 19.0%, and at a higher limit of about 6.0%, 7.0%, 8.0%, 9.0%, 10.0%, 11.0%, 12.0%, 13.0%, 14.0%, 15.0%, 16.0%, 17.0%, 18.0%, 19%, or about 20.0%, or any range that could be created between these recited ranges.

It has been found that at higher levels of water content in the fluid balance concentrate, the water activity of the fluid balance concentrate is such that the risk of unwanted growth of bacteria, yeasts, and mold increases. As such, in some aspects the fluid balance concentrate contains less than about 16.0% water and may contain water in an amount from about 12% to about 16%, or about 12.0%, 13.0%, 14.0%, 15.0%, or about 16.0%.

When the water is present in the fluid balance concentrate within the above-noted range from about 5.0% to about 20.0%, the fluid balance concentrate may exhibit a water activity less than about 0.50 and/or may have a water activity in a range between about 0.15 to about 0.49 or, in some instances, the fluid balance concentrate may exhibit a water activity of about 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, or about 0.49.

Further, since the fluid balance concentrate is a liquid, it would be desirable if it exhibited a viscosity suitable for further processing such as further processing to form a ready-to-drink fluid composition. With this in mind, the fluid balance concentrate exhibits a viscosity within a range between about 30 cP to about 300 cP at a temperature of about 70° F. In some instances, the fluid balance concentrate exhibits a viscosity, at a temperature of about 70° F., less than about 250 cP, or less than about 200 cP, or less than about 150 cP or less than about 100 cP. In some embodiments, the fluid balance concentrate exhibits a viscosity, at a temperature of about 70° F., between about 30 cP to about 100 cP or about 30 cP, 40 cP, 50 cP, 60 cP, 70 cP, 80 cP, 90 cP, or about 100 cP, any range that may be created with these values.

Further, it is noted that, in some instances, the fluid balance concentrate may exhibit a pH within a range of about 3.5 to about 4.5. For example, the fluid balance concentrate may exhibit a pH of about 3.5, 3.6, 3.7, 3.8, 3.9, 4.0, 4.1, 4.2, 4.3, 4.4, or about 4.5. In other embodiments, the fluid balance concentrate exhibits a pH within a range of about 4.0 to about 4.5 and a water activity in a range between about 0.15 to about 0.49.

Ready-to-Drink Fluid Compositions with the Fluid Balance Concentrate

It has been found that the above-described fluid balance concentrate may be useful in preparing ready-to-drink fluid compositions. Ready-to-drink refers to and means a fluid composition that can be consumed directly without any further preparation needed, like opening a can or bottle, essentially, it's ready to drink right out of the container. Such ready-to-drink fluid compositions may be useful for one or more of providing rapid hydration, and/or maintaining and/or improving bodily fluid balance, i.e., euhydration. To this end, without being bound by the following hypothesis, it is believed that the use of the described fluid balance concentrate may, when combined with sodium and/or electrolytes other than sodium, (a) provide an improved mouthfeel, particularly when compared to a ready-to-drink electrolyte-containing fluid composition with no fluid balance concentrate, (b) provide an organoleptic perception of mouth coating, (c) provide a perception of decreased bitterness and sweetness that may linger from the presence of non-nutritive sweeteners, when present, and/or (d) provide a perception of decreased saltiness and/or decreased perception of minerals.

The ready-to-drink fluid compositions may be provided in a container capable of containing and storing from about 300 ml to about 1,000 ml, although it is contemplated that greater or lesser sizes may be used. However, it has been found that generally consumers prefer a container suitable for containing and storing from about from about 300 ml to about 1,000 ml. Accordingly, the ready-to-drink fluid compositions may be provided in a container capable of containing and storing from about 300 ml, or about 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 ml.

With the above in mind, an exemplary ready-to-drink fluid composition may comprise, consist essentially of, or consist of water, an amount of the fluid balance concentrate to provide an amount of glycerol from about 0.01% to about 5.0% in the ready-to-drink composition, sodium, electrolytes other than sodium, a sweetener, and any optional additional ingredients. In some embodiments, the sweetener may be a non-nutritive sweetener. In some instances, the described ready-to-drink fluid composition may be free of carbohydrates.

With the above in mind, in this aspect, the ready-to-drink fluid composition may include water as a basic ingredient in the disclosed ready-to-drink fluid compositions, typically being the vehicle or primary liquid portion in which the remaining ingredients are dissolved, emulsified, suspended or dispersed. Purified water can be used in the manufacture of certain embodiments of the disclosed ready-to-drink fluid composition, and water of a standard beverage quality can be employed in order not to adversely affect beverage taste, odor, or appearance. The water typically will be clear, colorless, free from objectionable minerals, tastes and odors, free from organic matter, low in alkalinity and of acceptable microbiological quality based on industry and government standards applicable at the time of producing the ready-to-drink fluid composition.

In some exemplary embodiments, the water that may be used in the described ready-to-drink fluid composition and concentrates is “treated water,” which refers to water that has been treated to reduce the total dissolved solids of the water prior to optional supplementation, e.g., with calcium as disclosed in U.S. Pat. No. 7,052,725. Methods of producing treated water are known to those of ordinary skill in the art and include deionization, distillation, filtration and reverse osmosis (“RO”), among others. The terms “treated water,” “purified water,”, “demineralized water,” “distilled water,” and “RO water” are understood to be generally synonymous in this discussion, referring to water from which substantially all mineral content has been removed, typically containing no more than about 500 ppm total dissolved solids, e.g. 250 ppm total dissolved solids.

Water may be present in the ready-to-drink fluid composition in an amount from about 90% to about 99.9%, or from about 91%, 92%, 93%, 94%, 95%, 95.1%, 95.2%, 95.3%, 95.4%, 95.5%, 95.6%, 95.7%, 95.8%, 95.8%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or about 99.9%.

As mentioned above, the ready-to-drink fluid composition includes an amount of the fluid balance concentrate to provide an amount of glycerol from about 0.01% to about 5.0% in the ready-to-drink composition. In some aspects, the ready-to-drink fluid composition may include an amount of the fluid balance concentrate to provide an amount of glycerol in in the ready-to-drink fluid composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or about 5.0%.

It will be understood that by including the fluid balance concentrate in the ready-to-drink composition, the resulting ready-to-drink composition will contain a clouding agent, which may be present in the ready-to-drink composition in an amount from about 0.05% to about 0.5%, or about 0.06%, 0.07%, 0.08%, 0.09%, 0.1%, 0.2%, 0.3%, 0.4%, or about 0.5%.

The ready-to-drink fluid composition includes sodium in an amount from about 10 mmol/L of the ready-to-drink fluid composition to about 60 mmol/L. In some embodiments, the ready-to-drink fluid composition includes about 10 mmol/L, or about 15 mmol/L, 20 mmol/L, 25 mmol/L, 30 mmol/L, 35 mmol/L, 40 mmol/L, 45 mmol/L, 50 mmol/L, 55 mmol/L, or about 60 mmol/L of the ready-to-drink fluid composition

Sodium may be provided by sodium-containing compounds, such as, but not limited to sodium chloride, sodium lactate, trisodium citrate, sodium gluconate, monosodium phosphate, disodium phosphate, trisodium phosphate, tetrasodium acid pyrophosphate, sodium acid sulfate, sodium carbonate, sodium bicarbonate, sodium aspartate, sodium pyruvate, sodium acetate, sodium citrate, sodium citrate dihydrate or combinations thereof.

As noted above, the presence of glycerol as provided by the fluid balance concentrate will permit either (i) greater amounts of sodium to be included in the described ready-to-drink fluid compositions as compared to an identical ready-to-drink fluid composition where there is no glycerol present, i.e., where the glycerol has been replaced with water or (ii) the ready-to-drink fluid composition that contains both sodium and glycerol may require less sodium required to provide the same level of fluid balance as a ready-to-drink fluid composition that contains sodium but does not contain glycerol.

In the first instance, the presence of glycerol may attenuate the unpalatable salty taste resulting from a high concentration of sodium (i.e., sodium concentrations≥50 mMol). In the second instance, a fluid composition that contains both glycerol and sodium (denoted as fluid composition (a)) may be compared to an identical fluid composition that contains sodium but not glycerol, i.e., the glycerol has been replaced with sodium and water (denoted as fluid composition (b)). When the fluid balance of a human who has consumed fluid composition (a) is compared to the fluid balance of the same human who has consumed fluid composition (b), it has been found that to achieve the same fluid balance, the amount of sodium present in fluid composition (a) may be less than about 2 mmol/L to about 10 mmol/L than the amount of sodium present in fluid composition (b). In this regard, the reduced amount of sodium present in the glycerol-containing ready-to-drink fluid composition may be about 2 mmol/L, 3 mmol/L, 4 mmol/L, 5 mmol/L, 6 mmol/L, 7 mmol/L, 8 mmol/L, 9 mmol/L, or about 10 mmol/L.

Put another way, it has been found that with respect to achieving fluid balance, about 0.75% of glycerol will achieve about the same result as about 5 mmol/L of sodium.

Because both of the described ready-to-drink fluid compositions, i.e., composition containing the fluid balance concentrate and compositions not containing the fluid balance concentrate may contain electrolytes other than sodium, a sweetener, and any optional additional ingredients, those will be discussed below.

Ready-to-Drink Fluid Compositions without the Fluid Balance Concentrate

An exemplary fluid balance ready-to-drink fluid composition may comprise, consist essentially of, or consist of water, from about 0.01% to about 5.0% glycerol, sodium, electrolytes other than sodium, a sweetener, and any optional additional ingredients. In some embodiments, the sweetener may be a non-nutritive sweetener. In this embodiment, ready-to-drink fluid compositions are free of a clouding agent. In some instances, the described ready-to-drink fluid composition may be free of carbohydrates and/or clouding agent.

The ready-to-drink fluid compositions may be provided in a container capable of containing and storing from about 300 ml to about 1,000 ml, although it is contemplated that greater or lesser sizes may be used. However, it has been found that generally consumers prefer a container suitable for containing and storing from about from about 300 ml to about 1,000 ml. Accordingly, the ready-to-drink fluid compositions may be provided in a container capable of containing and storing from about 300 ml, or about 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 ml.

Such ready-to-drink fluid compositions may be useful for one or more of providing rapid hydration, and/or maintaining and/or improving bodily fluid balance, i.e., euhydration. To this end, without being bound by the following hypothesis, it is believed that the presence of glycerol may, when combined with sodium and/or electrolytes other than sodium, (a) provide an improved mouthfeel, particularly when compared to a ready-to-drink electrolyte-containing fluid composition with no glycerol, (b) will provide an organoleptic perception of mouth coating, (c) provide a perception of decreased bitterness and sweetness that may linger from the presence of non-nutritive sweeteners, when present, and/or (d) provide a perception of decreased saltiness and/or decreased perception of minerals.

With the above in mind, in this aspect, the ready-to-drink fluid composition may include water as a basic ingredient in the disclosed ready-to-drink fluid compositions, typically being the vehicle or primary liquid portion in which the remaining ingredients are dissolved, emulsified, suspended or dispersed. Purified water can be used in the manufacture of certain embodiments of the disclosed ready-to-drink fluid composition, and water of a standard beverage quality can be employed in order not to adversely affect beverage taste, odor, or appearance. The water typically will be clear, colorless, free from objectionable minerals, tastes and odors, free from organic matter, low in alkalinity and of acceptable microbiological quality based on industry and government standards applicable at the time of producing the ready-to-drink fluid composition.

In some exemplary embodiments, the water that may be used in the described ready-to-drink fluid composition and concentrates is “treated water,” which refers to water that has been treated to reduce the total dissolved solids of the water prior to optional supplementation, e.g., with calcium as disclosed in U.S. Pat. No. 7,052,725. Methods of producing treated water are known to those of ordinary skill in the art and include deionization, distillation, filtration and reverse osmosis (“RO”), among others. The terms “treated water,” “purified water,”, “demineralized water,” “distilled water,” and “RO water” are understood to be generally synonymous in this discussion, referring to water from which substantially all mineral content has been removed, typically containing no more than about 500 ppm total dissolved solids, e.g. 250 ppm total dissolved solids.

Water may be present in the ready-to-drink fluid composition in an amount from about 90% to about 99.9%, or from about 91%, 92%, 93%, 94%, 95%, 95.1%, 95.2%, 95.3%, 95.4%, 95.5%, 95.6%, 95.7%, 95.8%, 95.8%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or about 99.9%.

As mentioned above, the ready-to-drink fluid composition includes from about 0.01% to about 5.0% glycerol. In some aspects, the ready-to-drink fluid composition may include glycerol in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or about 5.0%.

The ready-to-drink fluid composition includes sodium in an amount from about 10 mmol/L of the ready-to-drink fluid composition to about 60 mmol/L. In some embodiments, the ready-to-drink fluid composition includes about 10 mmol/L, or about 15 mmol/L, 20 mmol/L, 25 mmol/L, 30 mmol/L, 35 mmol/L, 40 mmol/L, 45 mmol/L, 50 mmol/L, 55 mmol/L, or about 60 mmol/L of the ready-to-drink fluid composition

Sodium may be provided by sodium-containing compounds, such as sodium chloride, sodium lactate, trisodium citrate, sodium gluconate, monosodium phosphate, disodium phosphate, trisodium phosphate, tetrasodium acid pyrophosphate, sodium acid sulfate, sodium carbonate, sodium bicarbonate, sodium aspartate, sodium pyruvate, sodium acetate, sodium citrate, sodium citrate dihydrate or combinations thereof.

As noted above, the presence of glycerol will permit either (i) greater amounts of sodium to be included in the described ready-to-drink fluid compositions as compared to an identical ready-to-drink fluid composition where there is no glycerol present, i.e., where the glycerol has been replaced with water or (ii) the ready-to-drink fluid composition that contains both sodium and glycerol may require less sodium required to provide the same level of fluid balance as a ready-to-drink fluid composition that contains sodium but does not contain glycerol. For example, a fluid composition that contains both glycerol and sodium (denoted as fluid composition (a)) may be compared to an identical fluid composition that contains sodium but not glycerol, i.e., the glycerol has been replaced with sodium and water (denoted as fluid composition (b)). When the fluid balance of a human who has consumed fluid composition (a) is compared to the fluid balance of the same human who has consumed fluid composition (b), it has been found that to achieve the same fluid balance, the amount of sodium present in fluid composition (a) may be less than about 2 mmol/L to about 10 mmol/L than the amount of sodium present in fluid composition (b). In this regard, the reduced amount of sodium present in the glycerol-containing ready-to-drink fluid composition may be about 2 mmol/L, 3 mmol/L, 4 mmol/L, 5 mmol/L, 6 mmol/L, 7 mmol/L, 8 mmol/L, 9 mmol/L, or about 10 mmol/L.

Put another way, it has been found that with respect to achieving fluid balance, about 0.75% of glycerol will achieve about the same result as about 5 mmol/L of sodium.

Electrolytes Other than Sodium

Each of the described ready-to-drink fluid compositions may also contain electrolytes other than sodium. Other suitable non-sodium electrolytes may include one or more of potassium, magnesium, calcium, chloride, citrate, and phosphate. In some embodiments, the electrolytes other than sodium comprise, consist essentially of, or consist of electrolyte salts selected from potassium gluconate, calcium chloride (anhydrous), potassium chloride, potassium citrate, monopotassium phosphate, calcium lactate, magnesium oxide, or combinations thereof.

The electrolytes other than sodium may be present in each of the described ready-to-drink fluid compositions in a collective amount from about 0.0001% to about 0.7%. In this regard, the electrolytes other than sodium may be present in each of the described ready-to-drink fluid compositions in a collective amount from about 1 mmol/L to about 300 mmol/L or from about 10 mmol/L, 20 mmol/L, 30 mmol/L, 40 mmol/L, 50 mmol/L, 60 mmol/L, 70 mmol/L, 80 mmol/L, 90 mmol/L, 100 mmol/L, 110 mmol/L, 120 mmol/L, 130 mmol/L, 140 mmol/L, 150 mmol/L, 160 mmol/L, 170 mmol/L, 180 mmol/L, 190 mmol/L, 200 mmol/L, 210 mmol/L, 220 mmol/L, 230 mmol/L, 240 mmol/L, 250 mmol/L, 260 mmol/L, 270 mmol/L, 280 mmol/L, 290 mmol/L, or about 300 mmol/L of the ready-to-drink fluid compositions.

Potassium may be provided by potassium-containing compounds, such as potassium chloride, potassium citrate, potassium gluconate, monopotassium phosphate, dipotassium phosphate, tripotassium phosphate, tetrapotassium pyrophosphate, potassium sulfate, potassium acetate, potassium bicarbonate, potassium bromide, or combinations thereof. In some embodiments, the ready-to-drink fluid composition may include from about 1 mmol/L to about 30 mmol/L of potassium, or about 1 mmol/L, 2 mmol/L, 3 mmol/L, 4 mmol/L, 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, 20 mmol/L, 21 mmol/L, 22 mmol/L, 23 mmol/L, 24 mmol/L, 25 mmol/L, 26 mmol/L, 27 mmol/L, 28 mmol/L, 29 mmol/L, or about 30 mmol/L of potassium.

In some embodiments, each of the ready-to-drink fluid composition may include about 1 to 10 mEq/L of phosphate or about 3.6 to about 5.4 mEq/L of phosphate.

Magnesium may be provided by magnesium-containing compounds such as magnesium chloride, magnesium chloride hexahydrate, magnesium oxide, magnesium carbonate, magnesium sulfate, or a mixture thereof. In some embodiments, the ready-to-drink fluid composition may include from about 1 mmol/L to about 20 mmol/L of magnesium, or about 1 mmol/L, 2 mmol/L, 3 mmol/L, 4 mmol/L, 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, or about 20 mmol/L of magnesium.

Calcium may be provided by calcium-containing compounds such as anhydrous calcium chloride, calcium chloride, dibasic calcium phosphate, calcium lactate, calcium acetate, calcium citrate, calcium-D-gluconate, calcium levulinate, and mixtures thereof. In some embodiments, the ready-to-drink fluid composition may include from about 1 mmol/L to about 20 mmol/L of calcium, or about 1 mmol/L, 2 mmol/L, 3 mmol/L, 4 mmol/L, 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, or about 20 mmol/L of calcium.

Chloride may be provided by chloride-containing compounds, such as magnesium chloride, magnesium chloride hexahydrate, potassium chloride, anhydrous calcium chloride, or combinations thereof. In some embodiments, the ready-to-drink fluid composition may include about 4 to about 22 mEq/L of chloride. For example, the ready-to-drink fluid composition may include between about 7 to about 10 mEq/L of chloride.

### Sweeteners

While as noted above, the presence of glycerol may help in ameliorating potential palatability issues associated with high sodium concentrations, in some embodiments, it may be desirable to provide a sweetener, which may be a nutritive sweetener or, typically and more desirably, a non-nutritive sweetener.

The term “nutritive sweetener” refers generally to sweeteners which provide significant caloric content in typical usage amounts, e.g., more than about 5 calories per 8 oz. serving and may be considered to be a carbohydrate. In certain embodiments, the nutritive sweetener may include nutritive, natural crystalline or liquid sweeteners such as sucrose, liquid sucrose, fructose, liquid fructose, glucose, liquid glucose, leucrose, trehalose, galactose, isomaltulose, dextrose, maltodextrin, corn syrup solids, glucooligosaccharides, glucose-fructose syrup from natural sources such as apple, chicory, honey, etc., e.g., high fructose corn syrup, invert sugar, maple syrup, maple sugar, honey, brown sugar molasses, e.g., cane molasses, such as first molasses, second molasses, blackstrap molasses, and sugar beet molasses, sorghum syrup and/or others. The nutritive sweetener may be present in the ready-to-drink fluid composition in an amount from about 0.1% to about 10%, or about 0.25%, 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, or about 10%, or any range that can be created from these values.

The term “non-nutritive sweetener” refers to sweeteners that do not provide significant caloric content in typical usage amounts, i.e., is one which imparts less than 5 calories per 8 oz. serving. Non-nutritive artificial sweeteners suitable for at least certain exemplary embodiments include, for example, (i) rare sugars such as but not limited to D-allose, allulose (which also refers to psicose and/or D-psicose), L-ribose, D-tagatose, L-glucose, L-fucose, L-arabinose, D-turanose, D-leucrose, and mixtures thereof; (ii) peptide based sweeteners, e.g., aspartame, neotame, alitame and mixtures thereof; (iii) non-peptide based sweeteners, for example, sodium saccharin, calcium saccharin, acesulfame (including but not limited to acesulfame potassium), cyclamate (including but not limited to sodium cyclamate and/or calcium cyclamate), neohesperidin dihydrochalcone, sucralose, sorbitol, mannitol, xylitol, glycyrrhizin, neohesperidin dihydrochalcone, erythritol, meso-erythritol, maltitol, and mixtures thereof; (iv) maltose, lactose, fructo-oligosaccharides, Lo Han Guo powder, stevia, steviol glycosides, e.g., rebaudiosides such as Rebaudioside A, stevioside, etc., xylose, arabinose, isomalt, lactitol, maltitol, trehalulose, ribose, and mixtures thereof; (v) protein sweeteners such as monatin, thaumatin, monellin, brazzein, L-alanine, glycine related compounds, and mixtures thereof; and (vi) mixtures of any of (i) to (v).

The non-nutritive sweeteners described above may be present individually or collectively in an amount from about 0.1% to about 10% or about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or about 10% or any range that can be created from these values.

### Optional Additional Ingredients

The following describes optional additional ingredients that can be included in any of the described ready-to-drink fluid compositions. In other words, the following optional additional ingredients may be suitable for inclusion in the described ready-to-drink fluid compositions that contain the fluid balance concentrate or the described ready-to-drink fluid compositions that do not contain the fluid balance concentrate.

Non-mineral nutritive compounds such as vitamins may be added to the ready-to-drink fluid compositions. Examples of non-mineral nutritional supplement ingredients are known to those of ordinary skill in the art and include, for example, antioxidants and vitamins, including Vitamins A, D, E (tocopherol), C (ascorbic acid), B1 (thiamine), B2 (riboflavin), B6, B 12, and K, niacin, folic acid, biotin, and combinations thereof. The optional non-mineral nutritional supplements are typically present in amounts generally accepted under good manufacturing practices. Exemplary amounts are between about 1% and about 100% RDV, where such RDV are established. In certain exemplary embodiments the non-mineral nutritional supplement ingredient(s) are present in an amount of from about 5% to about 20% RDV, where established.

Silica may be included in any of the disclosed ready-to-drink fluid compositions. Without being bound by any theory, the inventors currently believe that the presence of silica in any of the disclosed ready-to-drink fluid compositions may improve hydration by enhancing how the body retains and utilizes water at the cellular level. Silica may be included in any of the disclosed ready-to-drink fluid compositions in any known form such as but not limited to silicon dioxide, silicic acid, and mixtures thereof. Silicic acids can be seen as hydrated forms of silica, namely 2H₂ₓSiOₓ₊₂=SiO₂·(H₂O)ₓ, and may include, but are not limited to, orthosilicic acid, metasilicic acid, pyrosilicic acid, disilicic acid, cyclo-tetrasilicic acid, and mixtures thereof.

The silica may be included in a range of about 0.1% to about 5.0% by weight of any of the disclosed ready-to-drink fluid compositions or about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or about 5%. Accordingly, the silica may be present at a lower limit of about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, or 4.9%, by weight of any of the disclosed ready-to-drink fluid compositions and may be present at an upper limit of about 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or about 5.0% by weight of any of the disclosed ready-to-drink fluid compositions or any range that may be created between any of the disclosed lower limit values and any of the disclosed upper limit values.

Acids may be included in any of the disclosed ready-to-drink fluid compositions and may serve any one or more of several functions, including, for example, providing antioxidant activity, lending tartness to the taste of the ready-to-drink fluid compositions, enhancing palatability, increasing thirst quenching effect, modifying sweetness and acting as a mild preservative by providing microbiological stability. Any suitable edible acidulant may be used, for example citric acid, malic acid, tartaric acid, phosphoric acid, ascorbic acid, lactic acid, formic acid, fumaric acid, gluconic acid, succinic acid, maleic acid, sodium acid sulfate and/or adipic acid. The acid may be used in solution form, for example, and in an amount sufficient to provide the desired pH of the ready-to-drink fluid compositions. Typically, for example, the one or more acids of the acidulant are used in an amount, collectively, of from about 0.01% to about 1.0% by weight of the ready-to-drink fluid compositions, e.g., from about 0.05% to about 0.5% by weight of the ready-to-drink fluid compositions, such as 0.1% to 0.25% by weight of the ready-to-drink fluid compositions, depending upon the acidulant used, desired pH, other ingredients used, etc.

Preservatives may be used in some embodiments of the disclosed ready-to-drink fluid compositions. That is, at least some exemplary embodiments contain an optional dissolved preservative system. Solutions with a pH below 4 and especially those below 3 typically are “micro stable,” i.e., they resist growth of microorganisms, and so are suitable for longer term storage prior to consumption without the need for further preservatives. However, an additional preservative system may be used if desired. If a preservative system is used, it may be added to the ready-to-drink fluid compositions at any suitable time during production, e.g., in some cases prior to the addition of the sweetener. As used here, the terms “preservation system” or “preservatives” include all suitable preservatives approved for use in food and beverage compositions, including, without limitation, such known chemical preservatives as benzoic acid, benzoates, e.g., sodium, calcium, and potassium benzoate, sorbates, e.g., sodium, calcium, and potassium sorbate, citrates, e.g., sodium citrate and potassium citrate, polyphosphates, e.g., sodium hexametaphosphate (SHMP), dimethyl dicarbonate, and mixtures thereof, and antioxidants such as ascorbic acid, EDTA, BHA, BHT, TBHQ, EMIQ, dehydroacetic acid, ethoxyquin, heptylparaben, and combinations thereof.

Preservatives may be used in amounts not exceeding mandated maximum levels under applicable laws and regulations. The level of preservative used typically is adjusted according to the planned final product pH, as well as an evaluation of the microbiological spoilage potential of the particular ready-to-drink fluid compositions. The maximum level employed typically is about 0.05% by weight of the ready-to-drink fluid compositions. It will be within the ability of those skilled in the art, given the benefit of this disclosure, to select a suitable preservative or combination of preservatives for ready-to-drink compositions according to this disclosure. In certain embodiments of the invention, benzoic acid or its salts (benzoates) may be employed as preservatives in the described ready-to-drink fluid compositions.

The described ready-to-drink fluid compositions may optionally include a flavor composition, for example, natural and synthetic fruit flavors, botanical flavors, other flavors, and mixtures thereof. In embodiments of the invention, flavors are present in an amount between 0 weight percent and 0.4 weight percent. As used here, the term “fruit flavor” refers generally to those flavors derived from the edible reproductive part of a seed plant. Included are both those wherein a sweet pulp is associated with the seed, e.g., banana, tomato, cranberry and the like, and those having a small, fleshy berry. The term berry also is used here to include aggregate fruits, i.e., not “true” berries, but that are commonly accepted as a berry. Also included within the term “fruit flavor” are synthetically prepared flavors made to simulate fruit flavors derived from natural sources. Examples of suitable fruit or berry sources include whole berries or portions thereof, berry juice, berry juice concentrates, berry purees and blends thereof, dried berry powders, dried berry juice powders, and the like.

Exemplary fruit flavors include the citrus flavors, e.g., orange, lemon, lime and grapefruit, and such flavors as apple, grape, cherry, and pineapple flavors and the like, and mixtures thereof. In certain exemplary embodiments, the ready-to-drink fluid compositions may include a fruit flavor component, e.g., a juice concentrate or juice. As used here, the term “botanical flavor” refers to flavors derived from parts of a plant other than the fruit. As such, botanical flavors may include those flavors derived from essential oils and extracts of nuts, bark, roots and leaves. Also included within the term “botanical flavor” are synthetically prepared flavors made to simulate botanical flavors derived from natural sources. Examples of such flavors include cola flavors, tea flavors, and the like, and mixtures thereof. The flavor component can further comprise a blend of various of the above-mentioned flavors. The particular amount of the flavor component useful for imparting flavor characteristics to the described ready-to-drink fluid compositions will depend upon the flavor(s) selected, the flavor impression desired, and the form of the flavor component. Those skilled in the art, given the benefit of this disclosure, will be readily able to determine the amount of any particular flavor component(s) used to achieve the desired flavor impression.

Other flavorings suitable for use in at least certain exemplary embodiments of the disclosed ready-to-drink fluid compositions include, e.g., spice flavorings, such as cassia, clove, cinnamon, pepper, ginger, vanilla spice flavorings, cardamom, coriander, root beer, sassafras, ginseng, and others. Numerous additional and alternative flavorings suitable for use in at least certain exemplary embodiments will be apparent to those skilled in the art given the benefit of this disclosure. Flavorings can be in the form of an extract, oleoresin, juice concentrate, bottler's base, or other forms known in the art. In at least certain exemplary embodiments, such spice or other flavors complement that of a juice or juice combination.

The one or more flavorings may be used in the form of an emulsion. A flavoring emulsion can be prepared by mixing some or all of the flavorings together, optionally together with other ingredients of the ready-to-drink fluid compositions, and an emulsifying agent. The emulsifying agent may be added with or after the flavorings mixed together. In certain exemplary embodiments the emulsifying agent is water-soluble. Exemplary suitable emulsifying agents include gum acacia, modified starch, carboxymethylcellulose, gum tragacanth, gum ghatti and other suitable gums. Additional suitable emulsifying agents will be apparent to those skilled in the art of ready-to-drink fluid compositions, given the benefit of this disclosure. The emulsifier in exemplary embodiments may comprise greater than about 3% of the mixture of flavorings and emulsifier. In certain exemplary embodiments the emulsifier is from about 5% to about 30% of the mixture of flavorings and emulsifier.

The described ready-to-drink fluid compositions may optionally include one or more color agents. In this regard, it may be appreciated that the above-described flavor composition may provide a color to the ready-to-drink fluid compositions and thus, an additional color agent may not be necessary or desired. When a color agent is included in the ready-to-drink fluid compositions it may be present in an amount from about 0.01% to about 1%.

### EXAMPLES

The invention will now be further elucidated with reference to the following examples, which should be understood to be non-limitative. It should be appreciated by those of ordinary skill in the art that the techniques disclosed in the examples that follow represent ones discovered by the inventors to function well in the practice of the invention and thus, constitute exemplary modes. One of ordinary skill in the art, when viewing this disclosure, will appreciate that many changes can be made in the specific embodiments while still obtaining similar or like results without departing from the spirit and scope of the present invention.

### Example 1

The following study sought to compare the effect on fluid balance at rest of consumption of 28 ounces of no, low, and moderate carbohydrate-containing drinks with high electrolyte concentration to a placebo. The study was a randomized, counterbalanced, crossover, placebo-controlled double-blind study with a complete block design. Accordingly, each subject ingested (drank) each of the ready-to-drink compositions and placebo but on different days. In other words, over a period of five different days, each subject drank either one of Compositions 1-4 or the placebo.

The following ready-to-drink compositions were prepared. The flavor and color of the placebo matched the test products (Compositions 1-4).

TABLE 1

| | | Composition | Composition | Composition | Composition |
|---|---|---|---|---|---|
| Ingredient | Placebo | 1 | 2 | 3 | 4 |
| water (wt. %) | 100 | balance | balance | balance | balance |
| glycerol (wt. %) | | 0 | 0.75 | 0.75 | 0.75 |
| sodium (mM) | | 45 | 40 | 40 | 40 |
| glucose (wt. %) | | | | 1.25 | |
| sucrose (wt. %) | | | | | 5.25 |

Twenty-one subjects entered the study and 24 hours prior to the ingestion of the ready-to-drink composition (compositions 1-4) or placebo, each subject was instructed to arrive well-hydrated (i.e., having a urine specific gravity less than 1.025). After collecting the urine sample, each subject was required to rest for 20 minutes, after which their body mass was measured and recorded.

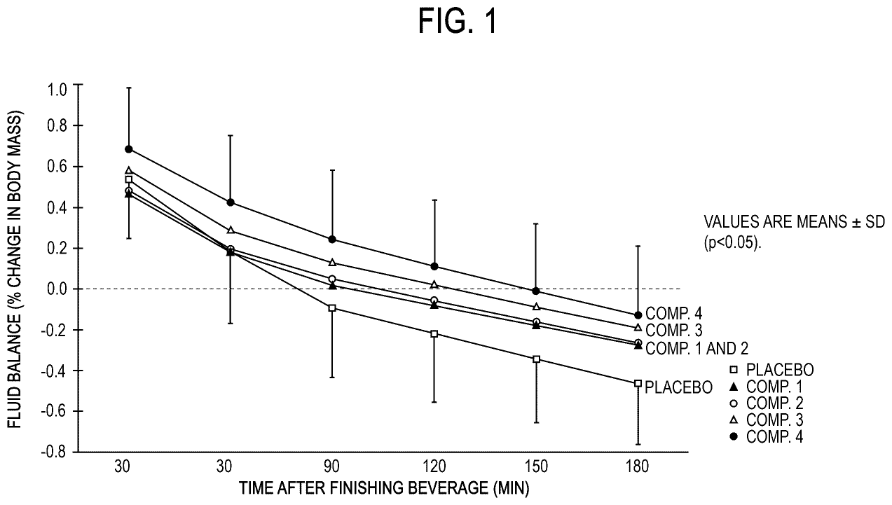

Each subject was provided with three aliquots of the assigned beverage so that the total amount of equaled 28 fluid ounces. Each subject was instructed to drink the first aliquot at t=−30 minutes, the second aliquot at t=−15 minutes, and the third and final aliquot at t=0 minutes. Thereafter, the body mass of each subject was measured every 30 minutes starting at 30 minutes after t=0 for 180 minutes. The results are shown in FIG. 1.

It will be appreciated from FIG. 1 that each of Compositions 1-4 exhibited better fluid balance and fluid retention than the placebo and it was found to be statistically significant (p<0.05). In addition, there was a statistically significant difference (p<0.05) between Composition 4 and each of Compositions 1-3 and the Placebo. Similarly, there was a statistically significant difference (p<0.05) between Composition 3 and each of Compositions 1, 2, and 4 and the Placebo. There was no statistically significant difference between Compositions 1 and 2 but there was a statistically significant difference (p<0.05) between Compositions 1 and 2 and each of Compositions 3, 4, and the Placebo.

With respect to Compositions 1 and 2 it will be appreciated that by providing glycerol (0.75%) the amount of sodium (40 mM) could be reduced compared to a beverage not containing glycerol and containing 5 mM more sodium (45 mM sodium vs. 40 mM sodium).

It will also be appreciated that the fluid balance and fluid retention significantly improved when the beverage included a carbohydrate. Without being bound by any specific theory, it is believed that the increase in the osmolality of the beverage due to the presence of the carbohydrate improved fluid retention.

### Example 2

The following study sought to compare the effect on fluid balance at rest of consumption of one liter of Composition 2 (ingredients noted above in Table 1) to a placebo (water with flavor and color to match Composition 2) over 8 hours. The study was a randomized, double blind, crossover study with two arms: test beverage versus placebo.

25 subjects entered the study and 24 hours prior to ingesting either Composition 2 or the placebo, each subject was instructed to arrive well-hydrated (i.e., having a urine specific gravity less than 1.025). After collecting the urine sample, each subject was required to rest for 10 minutes, after which their body mass was measured and recorded.

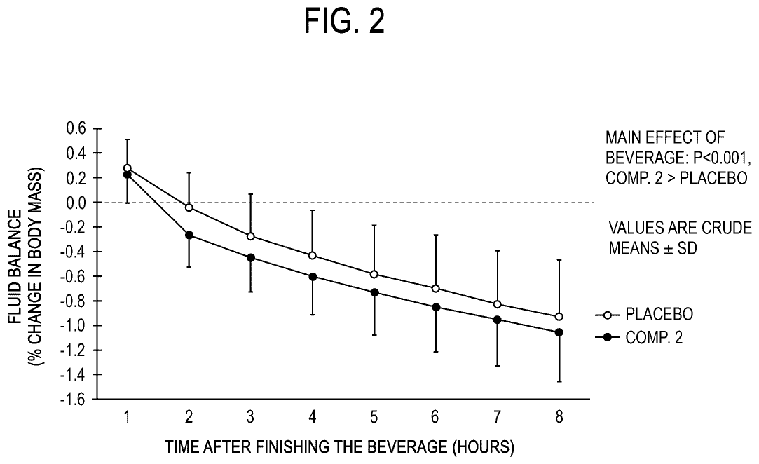

Each subject was provided with three aliquots of the assigned beverage so that the total amount equaled one liter. Each subject was instructed to drink the first aliquot at t=−30 minutes, the second aliquot at t=−15 minutes, and the third and final aliquot at t=0 minutes. Thereafter, the body mass of each subject was measured every 60 minutes starting at 60 minutes after t=0 for 480 minutes. The subjects were permitted to take a five-minute break after each body mass measurement. The results are shown in FIG. 2.

It can be seen that the overall net fluid balance was greater for Composition 2 than the placebo and the difference was statistically significant during the time period of 2 to 4 hours.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A fluid balance concentrate comprising glycerol, a clouding agent, and water.

2. The fluid balance concentrate of claim 1, wherein the glycerol is present in an amount from about 82% to about 92%.

3. The fluid balance concentrate of claim 2, wherein the clouding agent is present in an amount from about 3% to about 8%.

4. The fluid balance concentrate of claim 3, wherein water is present in an amount from about 5.0% to about 20.0%.

5. The fluid balance concentrate of claim 4, wherein the fluid balance concentrate exhibits a water activity less than 0.50.

6. The fluid balance concentrate of claim 4 wherein the fluid balance concentrate exhibits a water activity between about 0.15 and about 0.49.

7. The fluid balance concentrate of claim 6 wherein the fluid balance concentrate exhibits a viscosity between about 30 cP and about 300 cP at a temperature of about 70° F.

8. The fluid balance concentrate of claim 7 wherein the fluid balance concentrate exhibits a pH between about 3.5 and about 4.5.

9. A ready-to-drink fluid composition comprising:
(a) water;
(b) sodium;
(c) glycerol; and,
(d) electrolytes other than sodium, wherein the ready-to-drink composition is free of a clouding agent.

10. The ready-to-drink fluid composition of claim 9 wherein the glycerol is present in an amount from about 0.01% to about 5.0%.

11. The ready-to-drink fluid composition of claim 10 wherein a total amount of water is from about 90% to about 99.9%.

12. The ready-to-drink fluid composition of claim 11 wherein the sodium is present in an amount from about 10 mmol/L to about 60 mmol/L of the ready-to-drink fluid composition.

13. The ready-to-drink fluid composition of claim 12 wherein the electrolytes other than sodium include at least one of potassium, magnesium, calcium, chloride, citrate, phosphate, or mixtures thereof.

14. The ready-to-drink fluid composition of claim 13 wherein the electrolytes other than sodium include potassium in an amount from about 1 mmol/L to about 30 mmol/L.

15. The ready-to-drink fluid composition of claim 14 wherein the electrolytes other than sodium include magnesium in an amount from about 1 mmol/L to about 20 mmol/L.

16. The ready-to-drink fluid composition of claim 15 wherein the electrolytes other than sodium include calcium in an amount from about 1 mmol/L to about 20 mmol/L.

17. The ready-to-drink fluid composition of claim 16 wherein the electrolytes other than sodium include chloride in an amount from about 4 mEq/L to about 22 mEq/L.

18. The ready-to-drink fluid composition of claim 9, wherein the ready-to-drink fluid composition is free of carbohydrates.

19. The ready-to-drink fluid composition of claim 9 further comprising a sweetener selected from a nutritive sweetener, a non-nutritive sweetener, or combination thereof.

20. The ready-to-drink fluid composition of claim 19 wherein the sweetener is a nutritive sweetener that is selected from sucrose, fructose, glucose, leucrose, trehalose, galactose, isomaltulose, dextrose, maltodextrin, and mixtures thereof.

21. The ready-to-drink fluid composition of claim 20 wherein the nutritive sweetener is present in an amount from about 0.1% to about 10%.
